(19) United States
(12) Patent Application Publication
(10) Pub. No.: US 2008/0305158 A1
(43) Pub. Date: Dec. 11, 2008

(54) Methods For the Preparation of Stable Pharmaceutical Solid Dosage Forms of Atorvastatin and Amlodipine

(75) Inventors: Shrikant S. Chaudhari, Maharasthra (IN); Romi Barat Singh, Uttar Pradesh (IN); Vishnubhotla Nagaprasad, Andhra Pradesh (IN)

Correspondence Address: RANBAXY INC., 600 COLLEGE ROAD EAST, SUITE 2100, PRINCETON, NJ, 08540, US

(73) Assignee: RANBAXY LABORATORIES LIMITED, Gurgaon, Haryana (IN)

(21) Appl. No.: 11/722,620

(22) PCT Filed: Dec. 22, 2005
(86) PCT No.: PCT/IB2005/003857

§ 371 Date: Apr. 3, 2008

(30) Foreign Application Priority Data
Dec. 28, 2004 (IN) 201/DEL/2003

(51) Int. Cl. A61K 9/48; A61K 31/44; A61K 9/20; A61P 9/10; A61P 9/12

(52) U.S. Cl. 424/451; 514/356; 424/464

(57) ABSTRACT

The present invention relates to pharmaceutical compositions comprising amlodipine and pharmaceutically acceptable salts thereof, and atorvastatin and pharmaceutically acceptable salts thereof, and processes for their preparation.

### FIELD OF THE INVENTION

This invention relates to pharmaceutical compositions comprising amlodipine and pharmaceutically acceptable salts thereof, and atorvastatin and pharmaceutically acceptable salts thereof; and processes for their preparation. The invention also relates to kits containing such compositions as well as methods of using such compositions to treat subjects suffering from angina pectoris, atherosclerosis, combined hypertension and hyperlipidemia and/or hypercholesterolemia and to treat subjects presenting with symptoms of cardiac risk, including human subjects.

### BACKGROUND OF THE INVENTION

Atorvastatin, which is an inhibitor of the enzyme 3-hydroxy-3-methyl glutaryl coenzyme A reductase (HMG-CoA reductase), is commercially available for the treatment of primary hypercholesterolemia, dysbetalipoproteinemia and homozygous familial hypercholesterolemia.

Although cholesterol is an indispensable component of all cell membranes as well as a precursor of a variety of steroid hormones and bile acids, excessively high levels of blood cholesterol and lipids increase the risk of the onset of atherosclerosis and coronary heart disease. The blood cholesterol pool is generally dependent upon dietary uptake of cholesterol and the biosynthesis of cholesterol. HMG-CoA reductase enzyme inhibitors, such as atorvastatin, bring about a reduction in the levels of blood cholesterol, especially the low-density lipoproteins, by inhibiting the synthesis of cholesterol. They are therefore excellent candidates for controlling blood cholesterol levels.

Various attempts have been made to stabilize atorvastatin. WO 00/35425 discloses attempts to stabilize statin pharmaceutical compositions using buffering agents capable of providing a pH in the range from 7 to 11.

U.S. Pat. No. 5,686,104 and U.S. Pat. No. 6,126,971 discloses oral pharmaceutical compositions of atorvastatin in which the pharmaceutical composition is described as being stabilized by the addition of a pharmaceutically acceptable alkaline earth metal salt. According to these patents, large amounts of alkaline earth metal salt are required to stabilize the pharmaceutical composition. For example, these patents provide examples in which the drug compositions contain approximately 22% of an alkaline earth metal salt used to stabilize the atorvastatin. Nonetheless, these patents claim and/or state that between 5% and 75% of the composition can be the alkaline earth metal salt.

Amlodipine is a long acting calcium channel blocker marketed by Pfizer as amlodipine besylate under the trade name Norvasc®. It is available as oral tablets in strengths of 2.5 mg, 5 mg, and 10 mg, and is indicated for the treatment of hypertension, chronic stable angina and vasospastic angina. According to its labeling, the inactive ingredients in Norvasc® tablets include microcrystalline cellulose, dibasic calcium phosphate anhydrous, sodium starch glycolate, and magnesium stearate.

The preparation of amlodipine base is described in U.S. Pat. No. 4,572,909. Further, U.S. Pat. No. 4,879,303 discloses that free base compositions that include microcrystalline cellulose and dicalcium phosphate as diluents excessively stick to the tablet punches during processing and are not suitable in making solid pharmaceutical composition for peroral administration. The patent further discloses that the amlodipine besylate salt can be used to make solid pharmaceutical composition and those solid pharmaceutical compositions can include microcrystalline cellulose and dibasic calcium phosphate. The microcrystalline cellulose is present at between about 62% (w/w) and about 76% (w/w) of the total pharmaceutical composition. Subsequently, U.S. Patent Application No. 2003/0022922 discloses that to reduce the stickiness of the tablet, amlodipine free base should be incorporated into the tablet composition in the form of particulates having an average particle size of 150-350 μm; and a preferred excipient is a combination of calcium phosphate and microcrystalline cellulose. A capsule dosage form also is disclosed in this patent application as containing amlodipine base, microcrystalline cellulose, predried potato starch, and magnesium stearate. The microcrystalline cellulose makes up approximately 74% (w/w) of the capsule dosage form. The prior art literature discloses the use of dicalcium phosphate as one of the preferred excipients for amlodipine pharmaceutical composition. The inventors have discovered that the presence of dicalcium phosphate in the amlodipine pharmaceutical composition triggers the degradation of amlodipine, which is more pronounced at a pH below 6. Hence, the inventors have reason to believe that removal of dicalcium phosphate from the composition provides more stable pharmaceutical compositions of amlodipine.

Amlodipine is highly hygroscopic and absorbs moisture, which leads to degradation. One of the major routes of degradation is via the catalytic oxidative process, which is pH dependent. The major related substances produced are 13-ethyl-5-methyl-(4RS) 4-(2chlorophenyl)methyl[[2-[[2-(methylcarbamoyl)benzoyl]amino]ethoxy]methyl]-1,4dihydropyridine-3,5 dicarboxylate (“Impurity B”); 13-ethyl methyl[(2-aminoethoxy)methyl](2-chlorophenyl)methylpyridine-3,5 dicarboxylate (“Impurity D”); and 13ethyl 5-methyl(4RS) 4-(2-chlorophenyl)-2[[2-(1,3-dioxo-dihydro-2H-isoindolyl)ethoxy]methyl]methyl-1,4-dihydropyridine-3,5 dicarboxylate (“Impurity A”), along with some unknown impurities. Being an unstable compound, amlodipine requires well-directed stability approaches to formulate pharmaceutical compositions with reasonable stability.

PCT Application WO 99/11259 discloses therapeutic combinations comprising amlodipine and atorvastatin. It states that it is desirable to administer these two pharmaceutical agents to a patient in need of dual therapy.

PCT application WO 03/011283 discloses a composition of amlodipine and atorvastatin wherein atorvastatin granules and amlodipine blend are compressed into a tablet dosage form.

Our inventors have now developed alternative pharmaceutical compositions of amlodipine and atorvastatin. The pharmaceutical compositions obtained were found to be stable and have low impurity content.

### SUMMARY OF THE INVENTION

In one general aspect there is provided a process for making a pharmaceutical composition that includes amlodipine and atorvastatin or pharmaceutically acceptable salts thereof. The process includes: (a) blending atorvastatin and one or more pharmaceutically inert excipients; (b) blending amlodipine and one or more pharmaceutically inert excipients; (c) mixing the blends; (d) optionally granulating the blend; (e) lubricating the blend of step c) or granules of step d); and (f) filling the blend/granules into a capsule or compressing into tablet.

Embodiments of the process may include one or more of the following features. For example, the pharmaceutical composition may include less than about 0.5% impurity D of amlodipine.

The composition may further include one or more of diluents, binders, desiccants, disintegrants, coloring agents, flavoring agents, stabilizers, surfactants, lubricants/glidants and plasticizers. The stabilizers may be one or more of antioxidants, buffers, alkalinizing agents and chelating agents. The alkalinizing agents may be selected from one or more of sodium carbonate, sodium hydroxide, sodium silicate, disodium hydrogen orthophosphate, sodium aluminate and other alkali metal salts. The one or more alkalinizing agents may be present in the concentration at a range of about 1.2 to 5% by weight of the composition. The diluents may be one or both of microcrystalline cellulose and mannitol. The composition may be free of dicalcium phosphate.

The process may further include providing the pharmaceutical composition to treat subjects suffering from angina pectoris, atherosclerosis, combined hypertension and hyperlipidemia and/or hypercholesterolemia, by administering to the subject the pharmaceutical composition.

In another general aspect there is provided a process for making a pharmaceutical composition comprising amlodipine and atorvastatin or pharmaceutically acceptable salts thereof. The process includes steps of (a) preparing atorvastatin granules/blend and (b) preparation of amlodipine granules/blend. The steps of preparing the atorvastatin granules/blend include: (i) blending atorvastatin and one or more pharmaceutically inert excipients, (ii) optionally granulating the blend, and (iii) lubricating the granules/blend. The steps of preparing the amlodipine granules/blend include: (i) blending amlodipine and one or more pharmaceutically inert excipients, (ii) optionally granulating the blend, (iii) lubricating the granules/blend.

Embodiments of the process may include one or more of the following features. For example, the granules/blend of steps a) and b) may be compressed into a bilayer tablet or filled into a capsule.

The granules/blend of step b) and/or step a) may be compressed into a tablet. Further, the granules/blend of step b) and compressed tablet of step a) may be filled into capsules, or the granules/blend of step a) and compressed tablet of step b) may be filled into capsules.

The pharmaceutically acceptable salts of amlodipine may be besylate and the pharmaceutically acceptable salts of atorvastatin may be calcium or magnesium. The pharmaceutical composition may include less than about 0.5% impurity D of amlodipine.

The pharmaceutical composition may further include one or more of diluents, binders, desiccants, disintegrants, coloring agents, flavoring agents, stabilizers, surfactants, lubricants/glidants and plasticizers. The stabilizers may include antioxidants, buffers, alkalinizing agents and chelating agents. The alkalinizing agents may be one or more of sodium carbonate, sodium hydroxide, sodium silicate, disodium hydrogen orthophosphate, sodium aluminate and other alkali metal salts. The alkalinizing agents may be about 1.2 to 5% by weight of the composition. The composition is free of dicalcium phosphate.

The process may further include providing the pharmaceutical composition to treat subjects suffering from angina pectoris, atherosclerosis, combined hypertension and hyperlipidemia and/or hypercholesterolemia, by administering to the subject the pharmaceutical composition.

The details of one or more embodiments of the inventions are set forth in the description below. Other features, objects and advantages of the inventions will be apparent from the description and claims.

### DETAILED DESCRIPTION OF THE INVENTION

The pharmaceutical compositions described herein may be in various dosage forms, such as in the form of tablets or capsules.

In one of the embodiments, there is provided a process for the preparation of a pharmaceutical composition comprising atorvastatin and amlodipine or pharmaceutically acceptable salts thereof. The process includes the steps of
a) blending amlodipine besylate and one or more pharmaceutically inert excipients;
b) blending atorvastatin and one or more pharmaceutically inert excipients;
c) combining the blends of step a) and b)
d) optionally granulating the blend,
e) lubricating the blend or granules, and
f) compressing into suitable size solid dosage form or filling into capsules.

The pharmaceutical composition may be provided in the form of tablets wherein atorvastatin and amlodipine are processed to form a bilayer tablet.

In another embodiment, there is provided a process for the preparation of a pharmaceutical composition comprising atorvastatin and amlodipine or pharmaceutically acceptable salts thereof. The process includes the steps of:

i) preparation of atorvastatin granules
a) blending atorvastatin and one or more pharmaceutically inert excipients;
b) granulating the blend,
c) lubricating the granules,

ii) preparation of amlodipine granules
a) blending amlodipine and one or more pharmaceutically inert excipients;
b) granulating the blend,
c) lubricating the granules, and

iii) compressing the above two granules into a bilayer tablet.

The pharmaceutical composition may be provided in the form of capsules wherein one of the active ingredients is in the form of tablet and other in the form of the powder blend.

In another embodiment, there is provided a process for the preparation of a pharmaceutical composition comprising atorvastatin and amlodipine or pharmaceutically acceptable salts thereof. The process includes the steps of:
a) blending atorvastatin and one or more pharmaceutically inert excipients;
b) optionally granulating the blend,
c) lubricating the blend or granules,
d) compressing into suitable size tablet,
e) blending amlodipine and one or more pharmaceutically inert excipients; and
f) filling the tablet and amlodipine powder blend into a capsule.

In another embodiment, there is provided a process for the preparation of a pharmaceutical composition comprising atorvastatin and amlodipine or pharmaceutically acceptable salts thereof. The process includes the steps of:
a) blending amlodipine and one or more pharmaceutically inert excipients;
b) optionally granulating the blend,
c) lubricating the blend or granules,
d) compressing into suitable size tablet,
e) blending atorvastatin and one or more pharmaceutically inert excipients; and
f) filling the tablet and atorvastatin powder blend into a capsule.

In the above embodiments, a number of variations are possible. For example, the blend can be directly compressed or after granulation. Granules can be prepared by dry granulation or wet granulation. Tablets may contain a mixture of two blends or granules or these can be compressed separately as bilayered tablet.

The inventors have developed pharmaceutical composition of amlodipine and atorvastatin, which may be used for the treatment of subjects suffering from angina pectoris, atherosclerosis, combined hypertension and hyperlipidemia and/or hypercholesterolemia.

As used herein the term “atorvastatin” refers to atorvastatin calcium, atorvastatin magnesium, atorvastatin aluminum, atorvastatin iron, atorvastatin zinc, and other suitable salts of atorvastatin. Atorvastatin may exist any of the solid state forms available such as amorphous, crystalline or any other polymorphic form. Atorvastatin is present in the pharmaceutical composition at a dosage of between 1% to about 50% by weight of the composition.

In preparing the pharmaceutical compositions, atorvastatin of particle size d₉₀≦200 μm was found to be more favorable. These sizes were obtained either directly by the synthesis or by using conventional milling techniques, such as air jet milling, ball milling, cad milling, multi milling and other suitable size reduction techniques. The particle size of the atorvastatin was reduced to particle size d₉₀ of less than approximately 200 μm, and more particularly to particle size of between approximately 5 μm and 50 μm. The size of the particles was analyzed using a conventional particle size analyzer (e.g., a Malvern Master Sizer), although any conventional particle size analyzer is suitable.

In the above embodiments, the atorvastatin granules or blend may comprise one or more of stabilizing agents, antioxidants, diluents, disintegrants, surfactants and binders.

The stabilizing alkali metal salt additive is selected from amongst one or more of sodium carbonate, sodium hydroxide, sodium silicate, disodium hydrogen orthophosphate, sodium aluminate and other suitable alkali metal salts. In particular, the stabilizing alkali metal salt additive may be selected from amongst sodium carbonate and disodium hydrogen orthophosphate, although the other alkali metal salt additives may also be selected. The alkali metal salt additive is present at a concentration of between approximately 1.2% to a concentration that is less than about 5% by weight of the composition. In particular, better stabilization has been observed when the alkali metal salt additive is present at between approximately 1.5% and 4.8% by weight, and more particularly at between approximately 2.0% and 4.4% by weight. Suitable alkaline metal salt additives such as calcium carbonate, calcium hydroxide, magnesium carbonate, magnesium hydroxide, magnesium silicate, magnesium aluminate, and aluminum magnesium hydroxide in addition to alkali metal salt may also be used.

Amlodipine as used herein is the free base or besylate salt and can be of any form including, for example, crystalline form I, crystalline form II, amorphous form, and mixtures thereof. Amlodipine having a particle size d90 not more than 100 μm, more particularly d90 not more than 50 μm was found to be more favorable.

In the above embodiments, the amlodipine granules or blend may comprise one or more of stabilizing agents, diluents, disintegrants and binders.

The prior art literature discloses the use of dicalcium phosphate as one of the preferred excipients for amlodipine pharmaceutical composition. In our copending application WO 04/075825, it was disclosed that the presence of dicalcium phosphate in the amlodipine pharmaceutical composition triggers the degradation of amlodipine, which is more pronounced at a pH below 6. It was also observed that removal of dicalcium phosphate from the composition provides more stable pharmaceutical compositions of amlodipine. There was also observed improved stability by replacing dicalcium phosphate with microcrystalline cellulose and mannitol. The composition comprised less than 0.5% by weight of impurity D of amlodipine in the composition and has less than 2% by weight of total impurities of amlodipine in the composition. The stable pharmaceutical composition of amlodipine and atorvastatin includes microcrystalline cellulose and mannitol as diluents, but is substantially free of dicalcium phosphate. The impurity D of amlodipine is present in the composition in a concentration less than 0.5% by weight of the composition.

The term “dicalcium phosphate” as used herein includes anhydrous calcium phosphate, anhydrous dicalcium phosphate, dibasic calcium phosphate as well as hydrates and solvates thereof. The term “substantially free” as used herein refers to the use of dicalcium phosphate in a concentration less than that is used as a diluent.

Microcrystalline cellulose is a white, odorless, tasteless, free flowing powder, and is widely accepted in the pharmaceutical industry as a universal diluent. It is purified; partially depolymerized alpha cellulose derived from purified specialty grades of wood pulp. There are various grades which differ in bulk density, particle size, and moisture content. Some of the commercially available grades of microcrystalline cellulose are Avicel®, Vivapur® and Tabulos®. When used without mannitol, the amount of microcrystalline cellulose is increased relative to the prior art, e.g., greater than 80% (w/w) and, more particularly, greater than 90% (w/w). When used with mannitol, the amount of microcrystalline cellulose is greater than about 30% (w/w).

Mannitol is a naturally occurring sugar alcohol having a cool taste and 50% sweetness compared to sucrose. It is non-hygroscopic, chemically inert and does not undergo the Maillard reaction, and therefore does not discolor in the presence of free amines. Mannitol is available as powder and free flowing granules, and is used widely in pharmaceutical preparations. The granular form is particularly useful in direct compression technique of preparing tablets. Some of the commercial grades are Mannogem®, Pearlitol® and Partech M®. The concentration of mannitol may vary from about 2% to about 80%, in particular it may vary from 5% to 60% by weight of the total uncoated tablet weight.

The term “pharmaceutical composition” as used herein includes capsule, tablet or like.

The term “pharmaceutically inert excipient” as used herein includes substances known in the art as diluents, binders, desiccants, disintegrants, coloring agents, flavoring agents, stabilizers, surfactants, lubricants/glidants, plasticizers and preservatives for pharmaceutical compositions. The excipients are selected based on the desired physical aspects of the final tablets, e.g., obtaining a tablet with desired hardness and friability, being rapidly dispersible and easily swallowed, etc.

Examples of disintegrants include sodium starch glycolate, croscarmellose sodium, crospovidone, low substituted hydroxypropyl cellulose, and the like.

Examples of binders include methyl cellulose, hydroxypropyl cellulose, hydroxypropyl methylcellulose, polyvinylpyrrolidone, gelatin, gum arabic, ethyl cellulose, polyvinyl alcohol, pullulan, pregelatinized starch, agar, tragacanth, sodium alginate, and the like.

Examples of diluents include cellulose powdered, microcrystalline cellulose, dextrates, dextrins, dextrose excipients, fructose, kaolin, lactitol, lactose, mannitol, sorbitol, starch, starch pregelatinized, sucrose, sugar compressible, sugar confectioners, and the like.

Examples of lubricants and glidants include magnesium stearate, sodium stearyl fumarate, colloidal anhydrous silica, stearic acid, magnesium stearate, calcium stearate, talc, hydrogenated castor oil, sucrose esters of fatty acid, microcrystalline wax, yellow beeswax, white beeswax, and the like.

Examples of desiccants include colloidal silicon dioxide, silicon dioxide and the like.

Examples of surfactants include both non-ionic and ionic (Cationic, Anionic and Zwitterionic) surfactants suitable for use in pharmaceutical compositions. These include polyethoxylated fatty acids and their derivatives, for example polyethylene glycol 400 distearate, polyethylene glycol-20 dioleate, polyethylene glycol 4-150 mono dilaurate, and polyethylene glycol-20 glyceryl stearate; alcohol-oil transesterification products, for example polyethylene glycol-6 corn oil; polyglycerized fatty acids, for example polyglyceryl-6 pentaoleate; propylene glycol fatty acid esters, for example propylene glycol monocaprylate; mono and diglycerides, for example glyceryl ricinoleate; sterol and sterol derivatives, for example sitosterol; sorbitan fatty acid esters and their derivatives, for example polyethylene glycol-20 sorbitan monooleate and sorbitan monolaurate; polyethylene glycol 8 alkyl ether or phenols, for example polyethylene glycol-20 cetyl ether and polyethylene glycol 10-100 nonyl phenol; sugar esters, for example sucrose monopalmitate; polyoxyethylene polyoxypropylene block copolymers known as “poloxamer”; ionic surfactants, for example sodium caproate, sodium glycocholate, soy lecithin, sodium stearyl fumarate, propylene glycol alginate, octyl sulfosuccinate disodium, and palmitoyl carnitine; and the like.

Examples of plasticizers include polyethylene glycol, triethyl citrate, triacetin, diethyl plithalate, dibutyl sebacate and the like.

Examples of stabilizers include antioxidants, buffers, alkalinizing agents, chelating agents and the like.

The pharmaceutically acceptable antioxidants may be selected from amongst one or more of those suitable antioxidants known in the art. Examples of suitable pharmaceutically acceptable antioxidants include, but are not limited to, butylated hydroxyanisole (BHA), sodium ascorbate, butylated hydroxytoluene (BHT), sodium sulfite, citric acid, malic acid, and ascorbic acid.

The chelating agents may be selected from amongst one or more of those suitable chelating agents known in the art. Examples of suitable chelating agents include, but are not limited to, disodium edentate (EDTA). The chelating agents are present at a concentration of up to approximately 5% by weight of the pharmaceutical composition.

The coating may be selected from amongst one or more of those suitable coating materials known in the art. For example, the coating material can be Opadry or opadry AMB (aqueous moisture barrier).

Examples of coloring agents include any FDA approved colors for oral use.

In one of the embodiments, the pharmaceutical composition may be prepared by wet granulation technique, comprising the steps of blending drugs and optionally pharmaceutically inert excipient; granulating with a granulating fluid or solution/dispersion of binder; drying and sizing the granules; optionally blending with pharmaceutically inert extragranular excipients; lubricating the granules/blend; compressing the lubricated blend into suitable sized tablets; and optionally coating with film forming polymer and coating additives.

In another embodiment, the pharmaceutical composition may be prepared by dry granulation technique, comprising the steps of blending drugs and optionally pharmaceutically inert excipient; dry granulating the blend by roller compactor or slugging; lubricating the granules/blend; compressing the lubricated blend into suitable sized tablets; and optionally coating with film forming polymer and coating additives.

In another embodiment, the extended release tablets may be prepared by direct compression technique, comprising the steps of blending drugs and pharmaceutically inert excipient; lubricating the blend; directly compressing the lubricated blend into suitable sized tablets; and optionally coating with film forming polymer and coating additives.

In one of the embodiments, a pharmaceutical composition comprising atorvastatin and amlodipine is prepared by a process that includes the steps of:
a) dissolving antioxidants in an organic solvent,
b) granulating a portion of diluent with the above solution and drying the obtained granules,
c) blending alkali metal salt additive, diluent, lubricant/glidant, binder, disintegrant, amlodipine and atorvastatin, and
d) directly compressing the blend together.

The coating operation may be performed by applying one or more film forming polymers, with or without other pharmaceutically inert excipients, as a solution/suspension using any conventional coating technique known in the art, such as spray coating in a conventional coating pan or fluidized bed processor; or dip coating.

The following examples are provided to illustrate the invention but should not be construed as limiting the scope of the invention.

### EXAMPLES

| | Example 1 | Example 2 | Example 3 |
|---|---|---|---|
| Ingredients | (mg/tab) | (mg/tab) | (mg/tab) |
| Atorvastatin Calcium | 82.88 | 82.88 | 82.88 |
| Amlodipine Besylate | 13.87 | 13.87 | 13.87 |
| Mannitol | 80.00 | 80.00 | 80.00 |
| Pregelatinised Starch | 50.00 | 50.00 | 50.00 |
| Microcrystalline Cellulose | 443.15 | 459.15 | 427.29 |
| HPC-L | 16.00 | 16.00 | 16.00 |
| Sodium Carbonate Anhydrous | 25.00 | 25.00 | 25.00 |
| Colloidal Sillicon Dioxide | 16.00 | — | 16.00 |
| Croscarmellose Sodium | 48.00 | 48.00 | 48.00 |
| Sodium Lauryl Sulphate | 8.0 | 8.0 | 24.0 |
| Butylated Hydroxy Anisole | 4.7 | 4.7 | 4.56 |
| Butylated Hydroxy Toluene | 0.4 | 0.4 | 0.4 |
| Isopropyl Alcohol | q.s. | q.s. | q.s. |
| Magnesium Stearate | 12.00 | 12.00 | 12.00 |
| CORE TABLET WEIGHT | 800 mg | 800 mg | 800 mg |
| Opadry AMB | 32.00 | 32.00 | 32.00 |
| Purified water | q.s. | q.s. | q.s. |

Procedure:
1. Butylated hydroxyanisole and butylated hydroxyltoluene were dissolved in isopropyl alcohol.
2. About half of the quantity of the microcrystalline cellulose was passed through a screen and transferred to a rapid mixer granulator.
3. The solution of step 1 was added to the bulk of step 2 and mixed.
4. The wet mass was dried at 30° C.-40° C. in fluidized bed drier and passed through a sieve.
5. Sodium carbonate was milled and passed through sieve.
6. About one fourth of the quantity of the microcrystalline cellulose was passed through a multimill at slow speed.
7. Atorvastatin calcium, sodium stearyl fumarate and colloidal silicon dioxide were passed through a screen of quadro comil.
8. Amlodipine besylate and pregelatinised starch was passed together through a screen.
9. The remaining quantity of microcrystalline cellulose, hydroxypropyl cellulose and croscarmellose sodium was passed together through a screen.
10. Mannitol was passed through a screen.
11. The blends of steps 5-7 were mixed together.
12. The blends of step 8-9 were mixed together.
13. The blends of step 11 and 12 were mixed.
14. The blend of step 4 and mannitol of step 10 were mixed with the materials of step 13.
15. Magnesium stearate was passed through a screen and mixed with the materials of step 14.
16. The blend was compressed into tablets.
17. A dispersion of Opadry was made in purified water and coated onto the compressed tablet.

The composition of Example 3 was subjected to stability studies at 40° C./75% RH for 3 months and tested for assay, percent drug released at pH 6.8 buffer (USP type II and 900 ml buffer at 75 rpm) and related substances for amlodipine were measured as given in Table 1.

TABLE 1
Stability data for Example 3

| | | |
|---|---|---|
| Parameters | Initial | 40° C./75% RH-3 Month |

Assay (%)

| | | |
|---|---|---|
| Amlodipine | 97.2 | 94.7 |

Dissolution

| | Percent drug released | | | |
|---|---|---|---|---|
| | Initial | | 40° C./75% RH-3 Month | |
| | Amlodipine | Atorvastatin | Amlodipine | Atorvastatin |
| 15 minutes | 66 | 88 | 70 | 92 |
| 20 minutes | 71 | 93 | 70 | 94 |
| 30 minutes | 70 | 97 | 70 | 96 |

| Amlodipine besylate | Initial | 40° C./75% RH-3 Month |
|---|---|---|
| Impurity D | ND | ND |

While several particular forms of the inventions have been described, it will be apparent that various modifications and combinations of the inventions detailed in the text can be made without departing from the spirit and scope of the inventions. Accordingly, it is not intended that the inventions be limited, except as by the appended claims.

1. A process for making a pharmaceutical composition comprising amlodipine and atorvastatin or pharmaceutically acceptable salts thereof, the process comprising:
a) blending atorvastatin and one or more pharmaceutically inert excipients;
b) blending amlodipine and one or more pharmaceutically inert excipients;
c) mixing the blends;
d) optionally granulating the blend;
e) lubricating the blend of step c) or granules of step d); and
f) filling the blend/granules into a capsule or compressing into tablet.

2. The process of claim 1, wherein the pharmaceutical composition comprises less than about 0.5% impurity D of amlodipine.

3.-7. (canceled)

8. The process of claim 1, wherein the composition is free of dicalcium phosphate.

9. The process of claim 1, further comprising providing the pharmaceutical composition to treat subjects suffering from angina pectoris, atherosclerosis, combined hypertension and hyperlipidemia and/or hypercholesterolemia, by administering to the subject the pharmaceutical composition.

10. A process for making a pharmaceutical composition comprising amlodipine and atorvastatin or pharmaceutically acceptable salts thereof, the process comprising:
a) preparing atorvastatin granules/blend by
i) blending atorvastatin and one or more pharmaceutically inert excipients,
ii) optionally granulating the blend,
iii) lubricating the granules/blend; and
b) preparation of amlodipine granules/blend by
i) blending amlodipine and one or more pharmaceutically inert excipients,
ii) optionally granulating the blend,
iii) lubricating the granules/blend.

11. The process of claim 10, wherein the granules/blend of steps a) and b) are compressed into a bilayer tablet or filled into a capsule.

12. The process of claim 10, wherein the granules/blend of step b) and/or step a) is compressed into tablet.

13.-15. (canceled)

16. The process of claim 10, wherein the pharmaceutical composition comprises less than about 0.5% impurity D of amlodipine.

17.-20. (canceled)

21. The process of claim 10, wherein the composition is free of dicalcium phosphate.

22. The process of claim 10, further comprising providing the pharmaceutical composition to treat subjects suffering from angina pectoris, atherosclerosis, combined hypertension and hyperlipidemia and/or hypercholesterolemia, by administering to the subject the pharmaceutical composition.
